(19) United States
(12) Patent Application Publication
(10) Pub. No.: US 2017/0265455 A1
(43) Pub. Date: Sep. 21, 2017

(54) STORAGE MEDIA FOR TISSUE ALLOGRAFTS

(71) Applicant: ZIMMER ORTHOBIOLOGICS, INC., Austin, TX (US)

(72) Inventors: Ed E.C. Margerrison, Austin, TX (US); Hui Liu, Austin, TX (US); Rhonda Clarke, Cedar Park, TX (US)

(21) Appl. No.: 15/329,917

(22) PCT Filed: Jul. 23, 2015
(86) PCT No.: PCT/US2015/041717

§ 371 Date: Jan. 27, 2017

Related U.S. Application Data
(60) Provisional application No. 62/030,897, filed on Jul. 30, 2014.

(51) Int. Cl. A01N 1/02; A61F 2/28; A61F 2/30; A61L 27/38; A61L 27/36

(52) U.S. Cl. CPC A01N 1/0226; A61L 27/3817; A61L 27/3612; A61L 27/3608; A01N 1/0284; A61L 27/3852; A61L 27/3847; A61F 2/30756; A61F 2/28; A61L 2430/02; A61L 2430/06; A61F 2002/30224; A61F 2002/2839

(57) ABSTRACT

A fresh tissue allograft having at least one tissue portion maintained above a predetermined temperature to reduce the rate of cell death. A storage media having at least one free-radical scavenger is applied to the allograft to further slow the rate of cell death.

### CLAIM OF PRIORITY

This application claims the benefit of U.S. Provisional Patent Application Ser. No. 62/030,897, filed on Jul. 30, 2014, the benefit of priority of which is claimed hereby, and which is incorporated by reference herein in its entirety.

### TECHNICAL FIELD

This document pertains, but not by way of limitation, to methods of producing allografts for allotransplantation having an extended shelf life.

### BACKGROUND

Allotransplantation is the transplantation of allografts of donor tissue into a prepared site in corresponding tissue in the recipient to repair damaged tissue or supplement existing healthy tissue. Allografts typically comprise frozen tissue or “fresh”, unfrozen tissue in a storage media. While freezing the tissue allows the tissue to be safely stored for extended periods of time before being thawed and implanted, the freezing process kills the live cells in the tissue. In contrast, fresh tissue allografts contain live cells that can improve the likelihood that the allograft will successfully integrate with the surrounding tissue. However, the live cells in the fresh tissue allografts will naturally degrade overtime reducing the benefits provided by the live tissue allograft. The time period for implanting certain types of fresh tissue allografts with sufficient numbers of live cells to improved functionality and durability can be as short as one week. As allografts are produced from donor tissue, the limited supply and shelf life of live tissue allografts substantially weigh against the use of fresh tissue grafts despite the substantial benefit of improved functionality and durability.

Osteochondral allotransplantation is a common form of allotransplantation in which allografts containing cartilage and bone are transplanted to a recipient to repair or replace damaged cartilage. In particular, osteochondral allotransplantation is often used to repair lesions in the cartilage covering the condyle and cushioning the interface between the femur, tibia or patella. Each allograft for osteochondral allotransplantation typically includes a cartilage portion containing chondrocyte cells for repairing the cartilage and a bone portion for positioning and fixing the cartilage portion. An osteochondral allotransplantation procedure typically involves drilling or punching a hole through the lesion in the cartilage and into the underlying condyle bone to excise the lesion and a portion of the bone. The removal of the bone section provides a rigid mounting point for the allograft and can induce bleeding to promote in-growth of the surrounding bone into the bone portion of the allograft to fuse the allograft to bone. The allograft is shaped and sized to approximate the shape and depth of the bored out portion of the bone and cartilage before being inserted into the hole. During a successful allotransplantation, the surrounding healthy cartilage tissue will integrate with the cartilage tissue of the allograft.

Cartilage portions of osteochondral allografts can be particularly difficult to integrate with the surrounding tissue. Similarly, donor tissue for producing osteochondral allografts can be more limited than other tissue. Accordingly, the inability to effectively supply fresh tissue osteochondral allografts is particularly challenging.

### OVERVIEW

The present inventors have recognized, among other things, that a problem to be solved can include the high rate of cell death in live tissue allografts. In an example, the present subject matter can provide a solution to this problem, such as by applying a storage media containing at least one free-radical scavenger to the live tissue allograft. The free-radical scavenger can remove reactive elements in the allograft tissue and shield the allograft tissue from additional free radicals from entering the tissue during storage. The removed reactive elements and free radicals can reduce the rate of cell death, allowing implanting of the allograft tissue with a high level of live cells to improve functionality and durability of the allograft. The storage media can also include at least one additive for facilitating normal cellular activity to further reduce the rate of cell death.

In an example, a fresh tissue allograft having an extended shelf life can be prepared by a process including excising allograft tissue from a donor source. The process can further include maintaining the excised allograft tissue above a predetermined temperature and applying a storage media to the allograft tissue containing at least one free radical scavenger for reducing free-radicals in the storage media and the excised allograft tissue. The storage media and storage, according to an example of the present subject matter, can maintain viability of cells contained within the excised tissue such that at least 95% of the cells are viable after one week. Similarly, the storage media and storage, according to an example of the present subject matter, can maintain viability of cells contained within the excised tissue such that at least 70% of the cells are viable after four weeks. In an example, a method of transplanting a live tissue allograft can include providing allograft tissue from a donor source. The method can further include maintaining the allograft tissue above a predetermined temperature and applying a storage media to the allograft tissue containing at least one free radical scavenger for reducing free radicals in the storage media and the allograft tissue. The method can also include excising damaged tissue from recipient tissue to form a recipient site and inserting the allograft tissue into the recipient site.

In an example, a fresh tissue osteochondral allograft can include a cartilage portion and a bone portion. The osteochondral allograft can also include a storage media containing at least one free radical scavenger for reducing free-radicals. This overview is intended to provide an overview of subject matter of the present patent application. It is not intended to provide an exclusive or exhaustive explanation of the present subject matter. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view of an allograft having allograft tissue according to an example of the present subject matter.

FIG. 2 is a schematic diagram of the preparation of an allograft according to an example of the present subject matter.

FIG. 3 is a schematic diagram of the preparation of an allograft according to an example of the present subject matter.

FIG. 4 is a representative plot of cell viability over an extended storage duration of chondrocytes prepared according to various examples of the present subject matter as compared to chondrocytes stored in a conventional storage media.

FIG. 5 is a representative plot of cell viability over an extended storage duration of chondrocytes prepared according to various examples of the present subject matter as compared to chondrocytes stored in a conventional storage media.

FIG. 6 is a representative plot of cell viability an over extended storage duration of chondrocytes prepared according to various examples of the present subject matter as compared to chondrocytes stored in a conventional storage media.

FIG. 7 is a representative plot of cell viability over an extended storage duration of chondrocytes prepared according to various examples of the present subject matter containing different combinations of additives as compared to chondrocytes stored in a conventional storage media.

FIG. 8 is a representative plot of cell viability over an extended storage duration of chondrocytes prepared according to various examples of the present subject matter containing different combinations of additives as compared to chondrocytes stored in a conventional storage media.

FIG. 9 is a representative plot of cell viability over an extended storage duration of chondrocytes prepared according to various examples of the present subject matter containing different combinations of additives as compared to chondrocytes stored in a conventional storage media.

FIG. 10 is a representative plot of cell viability over an extended storage duration of cell viability over an extended storage duration of chondrocytes prepared according to an example of the present subject matter as compared to chondrocytes stored in a conventional storage media.

FIG. 11 is a representative plot of a ratio of live cells after an extended storage duration to live cells at the start of storage of chondrocytes prepared according to an example of the present subject matter as compared to chondrocytes stored in a conventional storage media.

FIG. 12 is a representative plot of a ratio of live cells after an extended storage duration to live cells at the start of storage of chondrocytes prepared according to an example of the present subject matter as compared to an chondrocytes stored in a conventional storage media.

FIG. 13 is a representative plot of a ratio of live cells after an extended storage duration to live cells at the start of storage of chondrocytes prepared according to an example of the present subject matter as compared to chondrocytes stored in a conventional storage media.

### DETAILED DESCRIPTION

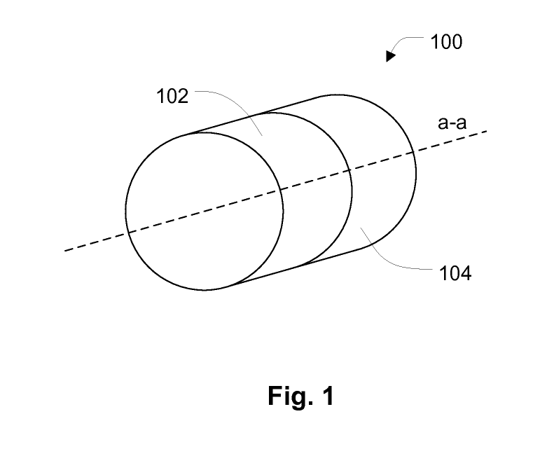

As depicted in FIG. 1, live allograft tissue 100 for allotransplantation and having an extended viable life, according to an example, includes a first tissue portion 102 and an applied storage media. The storage media reduces the death rate of live cells in at least the first tissue portion 102. A sufficiently large number of live cells in the first tissue portion 102 at implantation can contribute to improved functionality and durability of the allograft tissue 100 with the surrounding tissue at the recipient site for the allograft tissue 100. Certain types of allograft tissue 100 contain cells that, when implanted live, can continue to live in the recipient's body following transplantation, which can improved functionality and durability. Reducing the death rate of live cells in the first tissue portion 102 can increase the time period for which the allograft tissue 100 can be transplanted with improved functionality and durability provided by the live cells in the first tissue portion 102.

In an example, the first tissue portion 102 can comprise, but is not limited to, bone, bone marrow, cartilage, corneas, ligaments, intestines, whole or partial organs, skin, tendons or other tissues in which a quantity of the cells remain viable at implantation. In certain examples, allograft tissue 100 can comprise a second tissue portion 104, wherein the tissue of the second tissue portion 104 corresponds to the tissue of the first tissue portion 102. In an example, the second tissue portion 104 can comprise tissue that is ordinarily adjacent to the tissue of the first tissue portion 102. The second tissue portion 104 can be used to position or anchor the first tissue portion 102 during integration of the first tissue portion 102 into the tissue surrounding the recipient cite. In an example, the allograft tissue 100 can comprise osteochondral tissue, wherein the first tissue portion 102 comprises cartilage tissue and the second tissue portion 104 comprises bone. In this configuration, the first tissue portion 102 can contain chondrocytes that integrate with cartilage tissue surrounding the recipient site.

In an example, the storage media can comprise a free radical scavenger for reducing free radicals in at least the first tissue portion 102. Removing free radicals in the first tissue portion 102 can prevent undesirable reactions caused by the first tissue portion 102 that can result in an increased rate of cell death of live cells in the first tissue portion 102. Cell growth of new cells in the first tissue portion 102 effectively ceases after the first tissue portion 102 is excised from the donor tissue, which removes the live cells from the nutritional sources. The lack of nutritional sources also causes the live cells to die. Certain cell types, such as immunoprivileged chondrocytes, are capable of surviving for extended time periods following excising from the donor tissue, but can be prematurely damaged or die from reactions with free radicals. In an example, the free radical scavenger can comprise an oxygen radical scavenger or antioxidant for removing oxygen radicals from at least the first tissue portion 102. The oxygen radicals can cause oxidative stress in the live cells resulting in damage to the live cells and ultimately cell death. Scavenging the oxygen radicals can remove a substantial cause of cell death reducing the rate of cell death and extending the time period in which the first tissue portion 102 can be implanted into the recipient site with sufficient live cells to improve functionality and durability of the first tissue portion 102. In an example, the oxygen radical scavenger or antioxidant can include, but is not limited to, Vitamin C (ascorbic acid), Vitamin E (tocotrienols, tocopherols,), Carotenoids (carotenes, lycopene, lutein), Polyphenolic antioxidants (resveratrol, flavonoids), Glutathione, Selenium and N-Acetylcysteine.

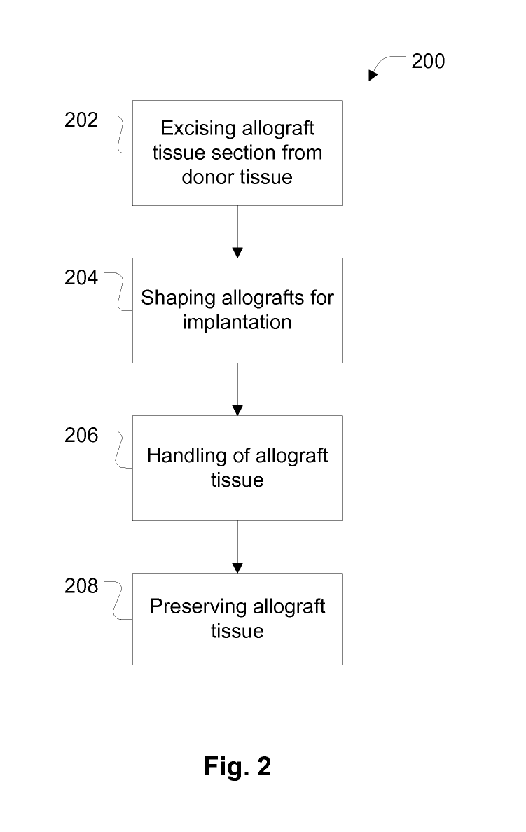

In an example, the storage media can comprise at least one additive for maintaining proper cellular function of live cells following excising for the first tissue portion 102. In certain examples, the additive can comprise a base medium or other energy source to support cellular metabolic activity or growth. The base medium or other energy source can include, but is not limited to, L-Alanyl-Glutamine, Dulbecco's modified Eagle's medium (“DMEM”), linoleic acid, sodium pyruvate and combinations thereof. In certain examples, the additive can prevent formation of undesirable byproducts, such as ammonia byproducts, that result from excising of the first tissue portion 102 or are ordinarily removed from the tissue by other processes. These additives can include, but are not limited to, L-Alanyl-L-Glutamine, transferrin, dexamethasone, N-Acetylcystenine and Glutathione. In certain examples, the additive can prevent programmed cell death (“PCD”) and can comprise, but is not limited to, resveratrol. In certain examples, the additive can supply nutrients and other chemicals for normal cellular function such as, but not limited to, amino acid and glucose uptake, cellular structure construction, intracellular transport, lipogenesis, nucleic acid and protein synthesis, and pH regulation. These additives can include, but are not limited to, hyaluronic acid, N-2-hydroxyelthlpiperazine-N′-2-Ethanesulfonic acid (“HEPES”), insulin, insulin-like growth factors, linoleic acid, selenium, and transferrin. As depicted in FIG. 2, a method 200 of preparing allograft tissue 100 having an extended viable life, according to an example, can comprise excising 202, shaping 204, handling 206 and preservation 208.

At 202, a portion of donor tissue can be excised to form a section of allograft tissue 100 containing at least one allograft. In an example, each allograft can be individually excised from the donor tissue. In another example, a segment of allograft tissue 100 can be excised from the donor tissue and subdivided into individual allografts. In this configuration, the segment of allograft tissue 100 can be divided such that each allograft includes tissue of the first tissue portion 102 and tissue of the second tissue portion 104 such as depicted in FIG. 1.

At 204, the allografts can be shaped for efficient implantation. In an example, each allograft can be formed into a cylindrical shape as depicted in FIG. 1 such that the allograft can be inserted into a cylindrical bore hole formed in the recipient site. The cylindrical shape can simplify the formation of the recipient site and conforming the shape and size of the allograft tissue 100 with the recipient site. In this configuration, the allograft tissue 100 can include a standard diameter, wherein the length of the allograft tissue 100 can be varied to fit the depth of the recipient site. In an example, each allograft of allograft tissue 100 can include at least a first tissue portion 102 and a second tissue portion 104, each allograft portion being shaped such that the first tissue portion 102 and the second tissue portion 104 align with the corresponding tissue upon insertion into the recipient site. In certain examples, each allograft can be shaped to correspond to the shape of the intended recipient site.

At 206, in an example, each allograft can be maintained above a predetermined temperature or range of temperatures, wherein the predetermined temperature corresponds to the freezing temperature of the allograft tissue 100. In an example, the tissue can be maintained at a temperature greater than 0° C. In another example, the tissue can be maintained at a temperature between about 0° C. to about 370° C. In yet another example, the tissue can be maintained at a temperature between about 19° C. to 26° C. While freezing the allograft tissue 100 will preserve the allograft tissue 100, the freezing of the tissue can result in the death of live cells in at least the first tissue portion 102. In an example, the first tissue portion 102 and the second tissue portion 104 can comprise different freezing temperatures, wherein the allograft is maintained above the greater freezing temperature.

At 208, storage media containing at least one free radical scavenger can be applied to each allograft portion. The storage media can be applied by a topical spray, liquid coating, submersion or other approaches for applying a liquid or semi-solid material to each allograft portion. In an example, the storage media can permeate the allograft to remove free radicals throughout the allograft.

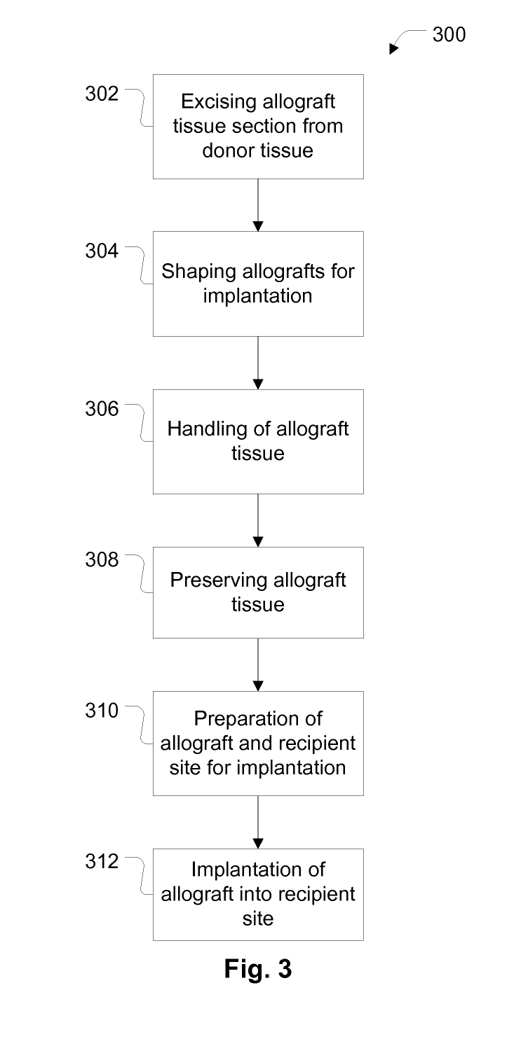

As depicted in FIG. 3, a method 300 of transplanting a live tissue allograft having an extended viable life, according to an example, can comprise excising 302, shaping 304, handling 306, preservation 308, preparation 310 and placement 312.

At 302, a portion of donor tissue can be excised to form a section of allograft tissue 100 containing at least one allograft. In an example, each allograft can be individually excised from the donor tissue. In another example, a segment of allograft tissue 100 can be excised from the donor tissue and subdivided into individual allografts. In this configuration, the segment of allograft tissue 100 can be divided such that each allograft includes tissue of the first tissue portion 102 and tissue of the second tissue portion 104 such as depicted in FIG. 1. In this configuration, the relative proportions of the first tissue portion 102 and second tissue portion 104 can correspond to the tissue proportions at the recipient site.

At 304, in an example, the allograft can be shaped into a predetermined shape corresponding to the desired recipient site. In certain examples, the recipient site can be formed as a bore hole comprising a standard shape, such as a cylindrical bore hole. In this configuration, each allograft can be formed into a cylindrical shape as depicted in FIG. 1 such that the allograft can be inserted into the cylindrical bore hole formed in the recipient site. The allograft can comprise a pre-determined diameter corresponding to a standard size for the bore hole at the recipient site. In this configuration, the allograft can be trimmed along axis a-a to adjust the length of the allograft to correspond to the depth of the bore hole. The corresponding diameter of the allograft and the bore hole can simplify and shorten the surgical implantation. In an example, each allograft of allograft tissue 100 can include at least a first tissue portion 102 and a second tissue portion 104, each allograft portion being shaped such that the first tissue portion 102 and the second tissue portion 104 align with the corresponding tissue upon insertion into the recipient site.

At 306, in an example, each allograft can be maintained above a predetermined temperature or range of temperatures, wherein the predetermined temperature corresponds to the freezing temperature of the allograft tissue 100. While freezing the allograft tissue 100 will preserve the allograft tissue 100, the freezing of the tissue can result in the death of live cells in the first tissue portion 102. In an example, the first tissue portion 102 and the second tissue portion 104 can comprise different freezing temperatures, wherein the allograft is maintained above the greater freezing temperature.

At 308, storage media containing at least one free radical scavenger can be applied to each allograft portion. The storage media can be applied by a topical spray, liquid coating, submersion or other approaches for applying a liquid or semi-solid material to each allograft portion. In an example, the storage media can permeate the allograft to remove free radicals throughout the allograft.

At 310, a recipient site can be prepared to receive the allograft. In an example, a bore hole can be cut into the damaged tissue or lesion to form the recipient site. In certain examples, the recipient site can comprise a cylindrical shaped bore hole surrounded by tissue corresponding to at least the first tissue portion 102. In an example, the bore hole can be cut to sufficient depth to expose an underlying tissue layer, wherein the underlying tissue layer corresponds to the second tissue portion 104. In an example, the length of the allograft can be trimmed to correspond to the depth of the bore hole.

At 312, in an example, the allograft can then be inserted into the recipient site such that a face of the allograft generally aligns with the tissue adjacent the recipient site. In an example, the allograft can be inserted such that the tissue of the first tissue portion 102 and the second tissue portion 104 align with corresponding tissue adjacent to the recipient site.

Each of these non-limiting examples can stand on its own, or can be combined in any permutation or combination with any one or more of the other examples.

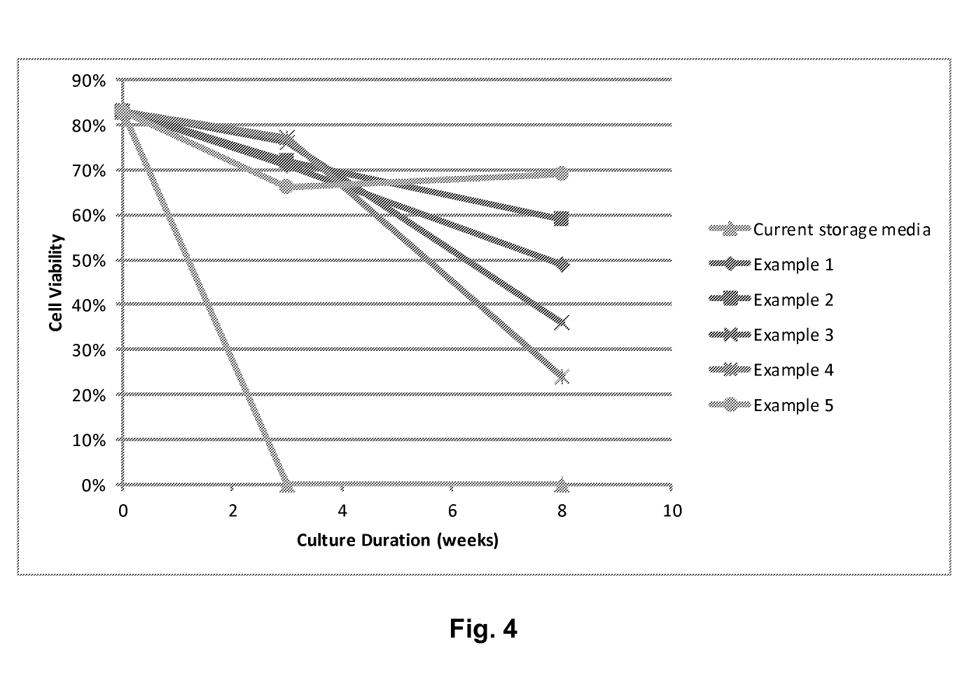

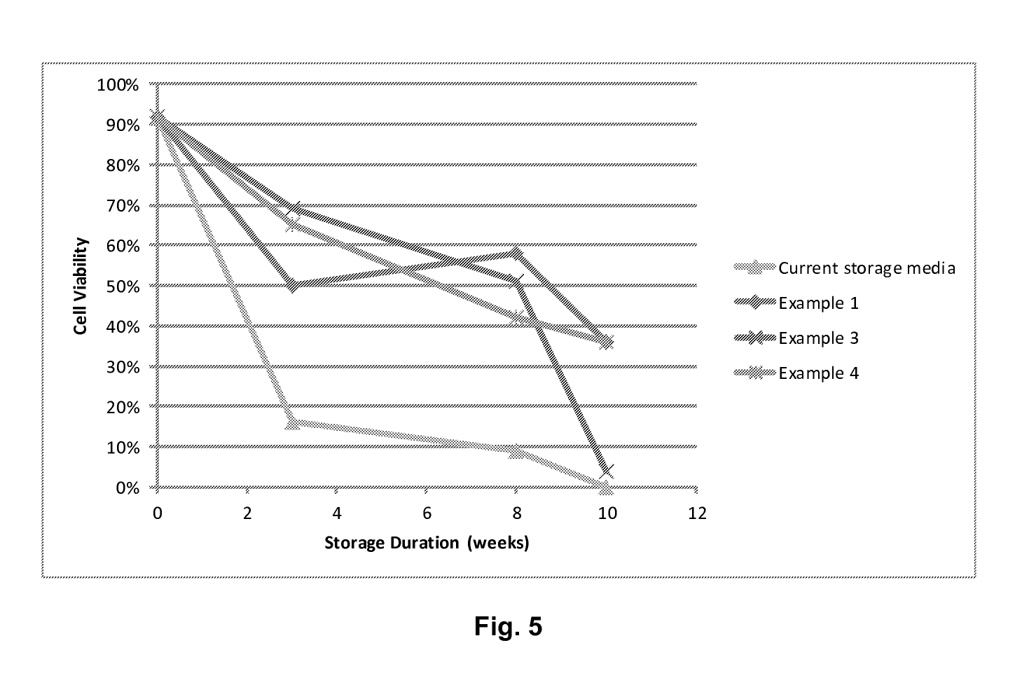

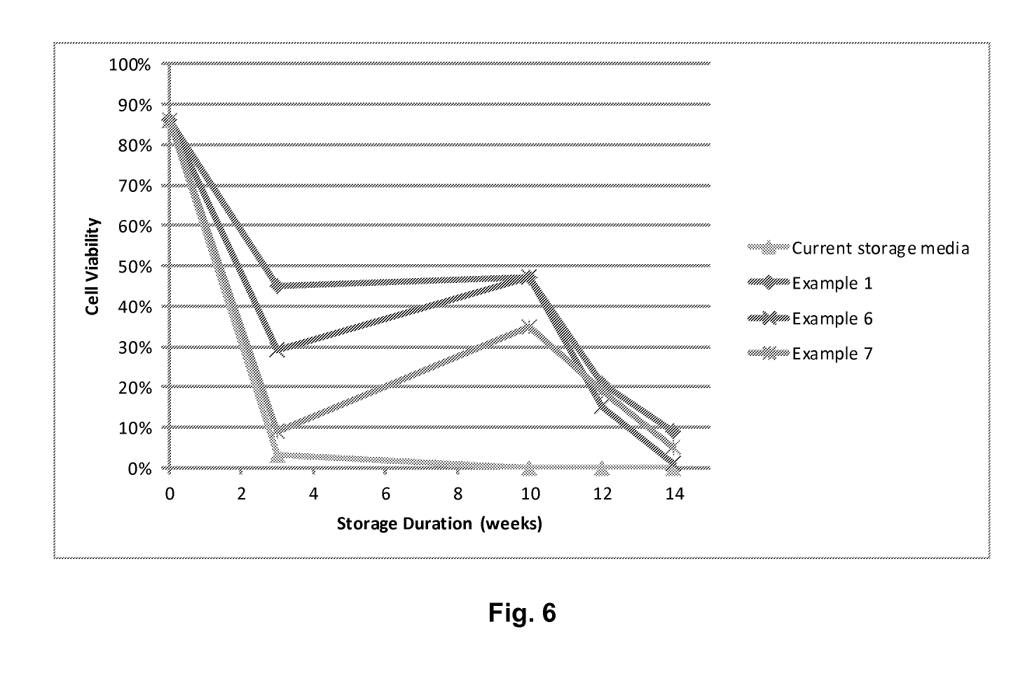

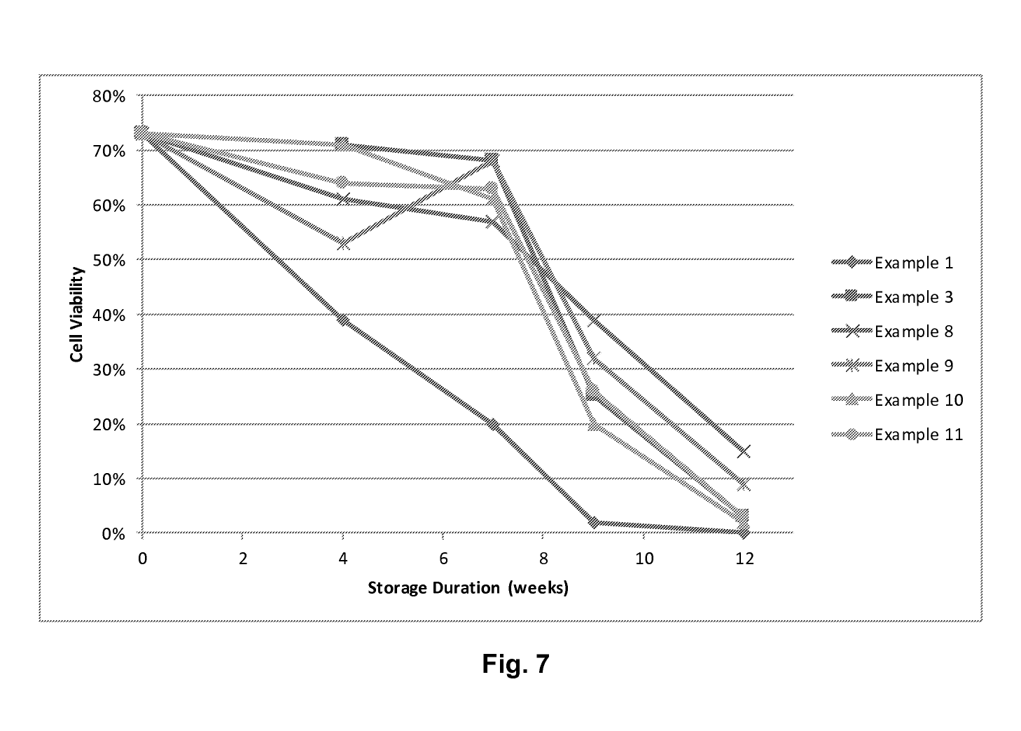

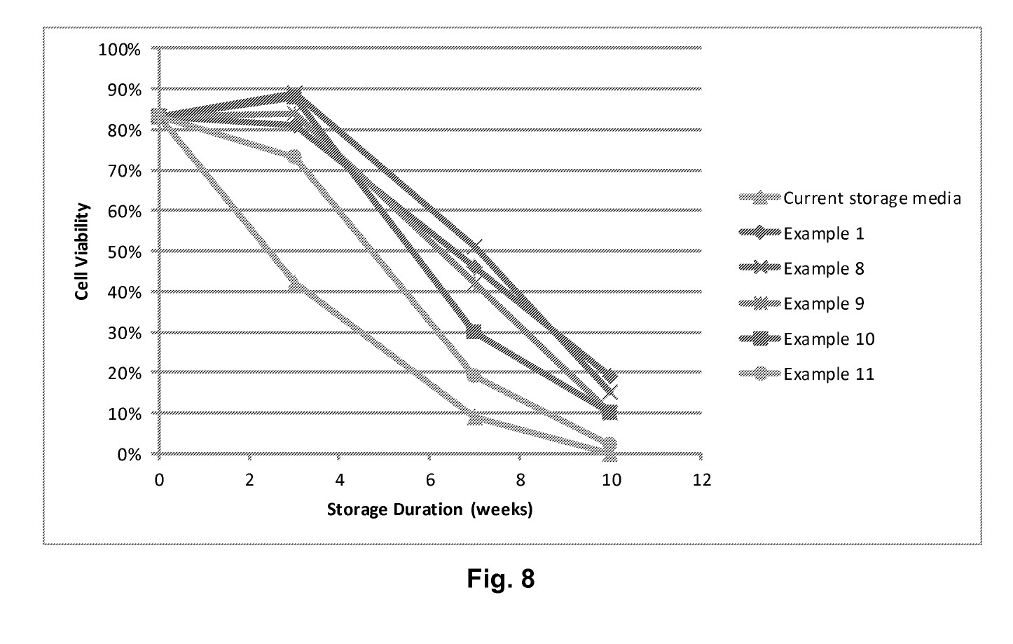

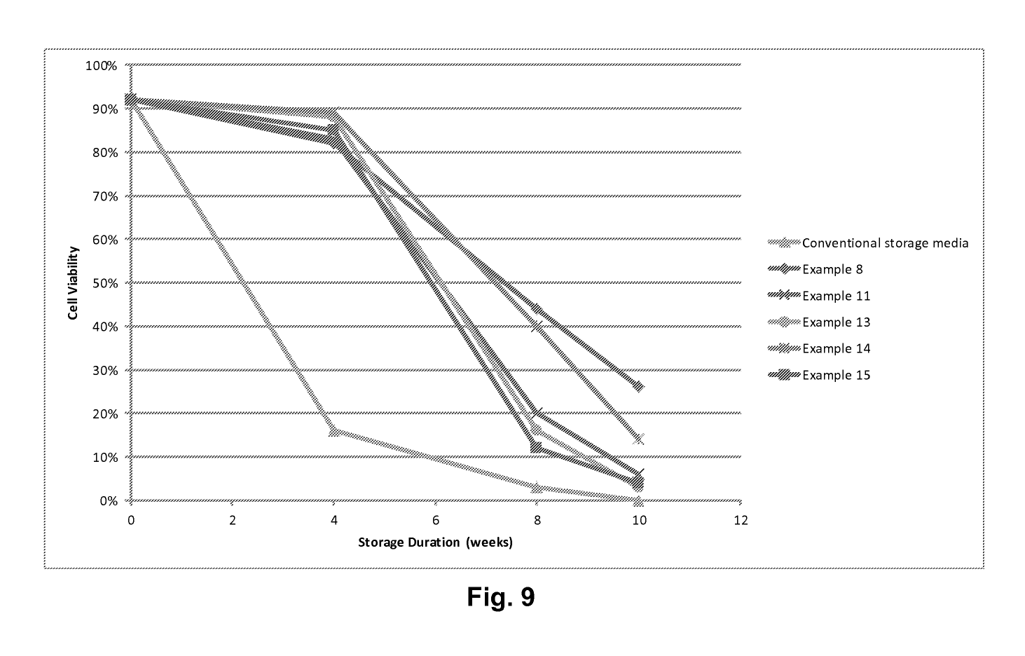

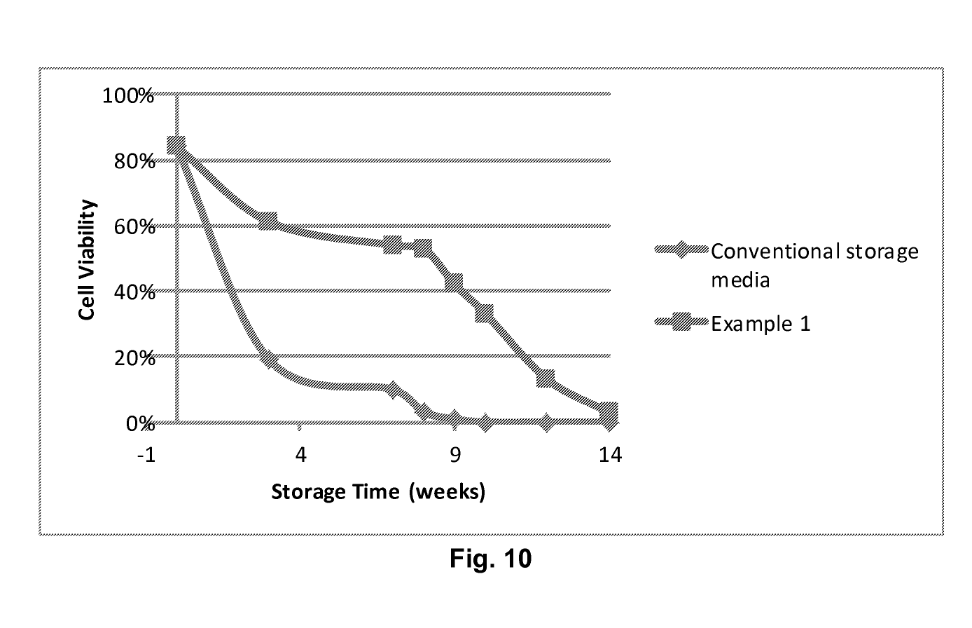

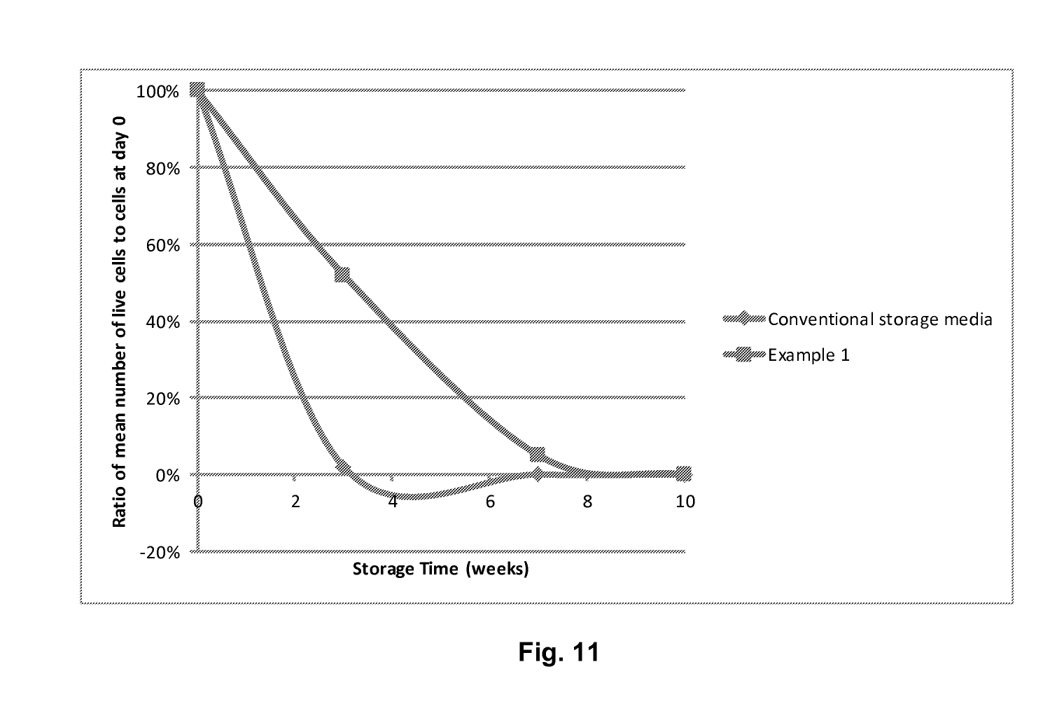

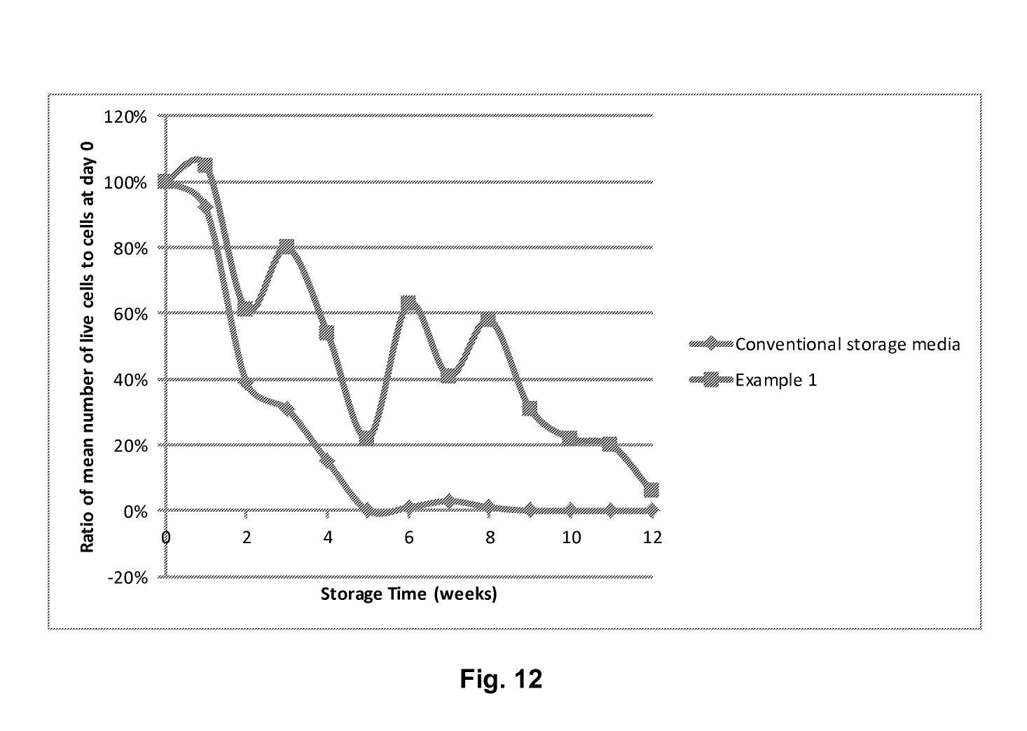

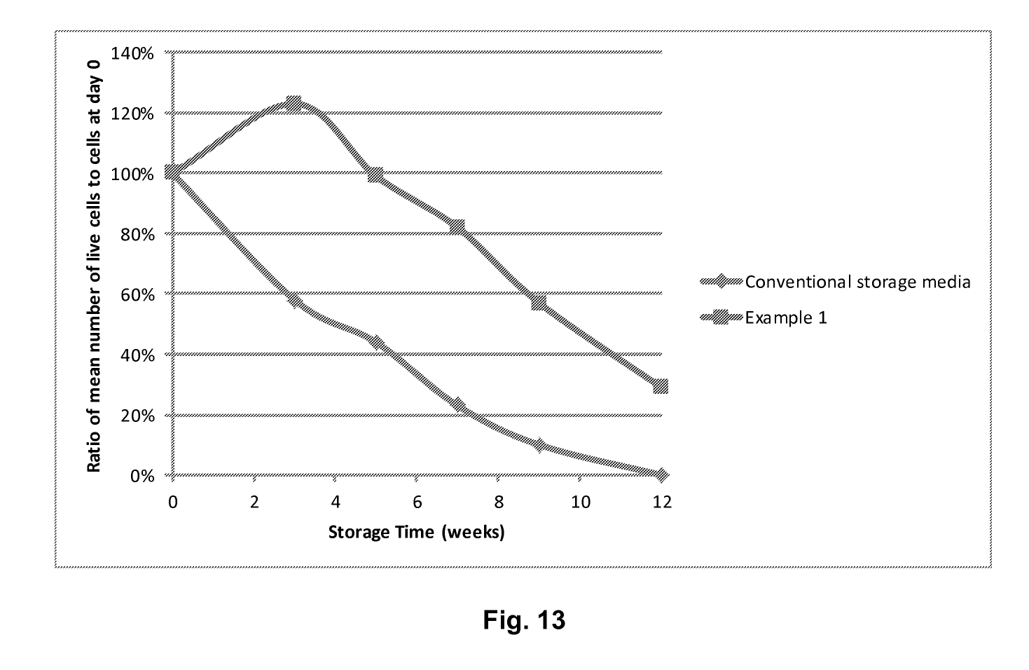

FIGS. 4-10 are representative plots of cell viability over an extended storage duration of chondrocytes prepared according to various Examples of the present subject matter as compared to chondrocytes stored in a conventional storage media. A conventional storage media as referenced in the present disclosure can include amino acids, vitamins, inorganic salts, carbohydrates and serum. As illustrated in FIGS. 4-10, the rate of cell death in a conventional storage media is about 10% of the total live cells per week. A storage media according to an example of the present disclosure can reduce cell death to at least about 5% of the total live cells per week. FIGS. 11-13 are representative plots of a ratio of live cells after an extended storage duration to live cells at the start of storage of an cartilage allograft prepared according to various Examples of the present subject matter as compared to an allograft stored in a conventional storage media. The various Examples depicted in FIGS. 4-13 are described in further detail below.

Example 1 can include allograft tissue to which a storage media including at least one free radical scavenger is applied. In an example, the at least one free radical can include an oxygen free-radical scavenger such as N-Acetyl-L-Cysteine.

Example 2 can include the formulation of Example 1 and further include at least one antibiotic. In an example, the antibiotic can comprise penicillin streptomycin.

Example 3 can include the formulation of Examples 1 or 2 and further include a serum-free serum replacement for growing and maintaining cells in culture. In an example, the serum replacement can comprise KNOCK OUT SERUM REPLACEMENT produced by LIFE TECHNOLOGIES of Carlsbad, Calif.

Example 4 can include the formulation of Example 3 and further include a second free-radical scavenger. In an example, the second free-radical scavenger can comprise methylene blue.

Example 5 can include the formulation of any one of the proceeding Examples and further include a second free-radical scavenger. In an example, the second free-radical scavenger can comprise methylene blue.

Example 6 can include the formulation of any one of the proceeding Examples, wherein the at least one free-radical scavenger can comprise a reduced concentration.

Example 7 can include the formulation of any one of the proceeding Examples and further include at least one anti-apoptotic agent. In an example, the at least one apoptotic agent can comprise hyaluronic acid.

Example 8 can include the formulation of any one of the proceeding Examples and further include at least a second free radical scavenger. In an example, the second free radical scavenger can comprise vitamin E.

Example 9 can include the formulation of Example 8 and further include at least one anti-apoptotic agent. In an example, the at least one anti-apoptotic agent can comprise insulin-like growth factor.

Example 10 can include the formulation of any one of proceeding Examples 9-10 and further include a serum-free serum replacement. In an example, the serum replacement can include KNOCK OUT SERUM REPLACEMENT.

Example 11 can include the formulation of any one of proceeding Examples 8-10 and further include a serum-free serum replacement. In an example, the serum replacement can include KNOCK OUT SERUM REPLACEMENT.

Example 12 can include the formulation of any one of the proceeding Examples, wherein the concentration of the at least one free-radical scavenger is doubled. In an example, the at least one free radical can include an oxygen free-radical scavenger such as N-Acetyl-L-Cysteine.

Example 13 can include the formulation of Example 12 and further include a serum replacement and a second free radical scavenger. In an example, the serum replacement can comprise KNOCK OUT SERUM REPLACEMENT and the second free-radical scavenger can comprise vitamin E.

Example 14 can include the formulation of any one of proceeding Examples 8-13 and further include a third free-radical scavenger. In an example, third free-radical scavenger can comprise Resverartol 20.

Example 15 can include the formulation of any one of proceeding Examples 8-14 and further include a third free-radical scavenger. In an example, third free-radical scavenger can comprise Resverartol 100.

As depicted in FIGS. 4-10, allografts having a storage media according to an example of the present subject matter can have improved cell viability over time as compared to conventional storage media. As depicted in FIGS. 11-13, allografts having a storage media according to an example of the present subject matter can have a higher ratio of live cells after an extended duration to live cells at the initial live cell count than allografts having conventional storage media.

Each of these non-limiting Examples can stand on its own, or can be combined in any permutation or combination with any one or more of the other Examples.

The above detailed description includes references to the accompanying drawings, which form a part of the detailed description. The drawings show, by way of illustration, specific embodiments in which the present subject matter can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the present subject matter should be determined with reference to the appended claims, along with the full scope of equivalents to which such claims are entitled.

1. An allograft having an extended shelf life prepared by a process comprising:
excising a quantity of allograft tissue from a donor source;
maintaining the excised allograft tissue above a predetermined temperature; and
applying a storage media to the excised allograft tissue, the storage media containing at least one free radical scavenger for reducing free-radicals in the excised allograft tissue.

2. The allograft of claim 1, wherein the donor source comprises osteochondral tissue having a bone portion and a cartilage portion;
wherein the excised allograft tissue contains a quantity of live chondrocytes from the cartilage portion.

3. The allograft of claim 2, wherein death of live cells in the allograft tissue dies at a rate less than about 5% per week.

4. The allograft of claim 1, wherein the excised allograft tissue contains a quantity of live cells;
wherein at least about 95% of live cells are viable after a week.

5. The allograft of claim 1, wherein the excised allograft tissue contains a quantity of live cells;
wherein at least about 70% of live cells are viable after 4 weeks.

6. The allograft of claim 1, further comprising:
shaping the excised allograft tissue into a cylindrical shape.

7. The allograft of claim 1, wherein the predetermined temperature corresponds to a freezing temperature for the allograft tissue.

8. The allograft of claim 1, wherein the at least one free radical scavenger comprises an oxygen free radical scavenger for reducing oxygen free radicals in the storage media and the excised allograft tissue.

9. A method of transplanting a live tissue allograft, comprising:
receiving allograft tissue from a donor source;
maintaining the allograft tissue above a predetermined temperature;
applying a storage media to the allograft tissue, the storage media comprising at least one free radical scavenger for reducing free radicals in the allograft tissue;
excising damaged tissue from recipient tissue to form a recipient site in the recipient tissue; and
inserting the allograft tissue into the recipient site.

10. The allograft of claim 9, wherein the donor source comprises osteochondral tissue having a bone portion and a cartilage portion;
wherein the excised allograft tissue contains a quantity of live chondrocytes from the cartilage portion.

11. The allograft of claim 10, wherein the rate of death of live cells in the allograft tissue is less than about 5% per week.

12. The method of any one of claim 10, further comprising:
aligning the bone portion and the cartilage portion of the allograft tissue with corresponding tissue at the recipient site.

13. The method of claim 12, wherein the excised allograft tissue includes a quantity of live cells, wherein the live cells integrate with corresponding cells at the recipient site.

14. The method of claim 9, wherein death of live cells in the allograft tissue dies at a rate less than about 5% per week.

15. The method of claim 9, further comprising:
shaping the excised allograft tissue into a cylindrical shape, wherein the recipient site comprises a cylindrical bore hole.

16. The method of claim 9, further comprising:
measuring a depth of the recipient site; and
cutting the allograft tissue to a length corresponding the measured depth of the recipient site.

17. The method of claim 9, wherein the predetermined temperature corresponds to a freezing temperature for the allograft tissue.

18. The method of claim 9, wherein the at least one free radical scavenger comprises an oxygen free radical scavenger for reducing oxygen free radicals in the storage media and the excised allograft tissue.

19. A fresh tissue osteochondral allograft, comprising:
a cartilage portion, containing a quantity of live chondrocytes;
a bone portion; and
a storage media containing at least one free radical scavenger for reducing free-radicals in the cartilage portion and the bone portion.

20. The fresh tissue osteochondral allograft of claim 19, wherein the allograft comprises a temperature greater than a predetermined threshold temperature, wherein the predetermined threshold temperature corresponds to a freezing temperature for the cartilage portion.

21. The fresh tissue osteochondral allograft of claim 19, wherein at least 95% of chondrocytes are viable after a week.

22. The fresh tissue osteochondral allograft of claim 19, wherein at least 70% of chondrocytes are viable after four weeks.
